(19) United States
(12) Patent Application Publication
(10) Pub. No.: US 2021/0097558 A1
(43) Pub. Date: Apr. 1, 2021

(54) SYSTEMS AND METHODS FOR COUPON-BASED INCENTIVE PROMOTIONS AND TRACKING

(71) Applicant: Luvo Inc., Blaine, WA (US)

(72) Inventors: Christine Day, Point Roberts, WA (US); Mark Walker, Austin, TX (US); Lawrence Scotland, North Vancouver (CA)

(73) Assignee: Luvo Inc., Blaine, WA (US)

(21) Appl. No.: 17/039,820

(22) Filed: Sep. 30, 2020

Related U.S. Application Data
(60) Provisional application No. 62/908,500, filed on Sep. 30, 2019.

(51) Int. Cl. G06Q 30/02

(52) U.S. Cl. CPC G06Q 30/0203; G06Q 30/0217; G06Q 30/0201; G06Q 30/0222

(57) ABSTRACT

A promotions system and methods are provided for generating incentive-based promotions for specialized products with subsequent generated tracking metric data. The promotions system is configured to receive promotion data, generate coupon data based on the promotion data, and transmit the coupon data to a retail server. The generated coupon data generally comprises approved products list data and medically tailored meals data. The retail server checks purchased products against the approved products list data and the medically tailored meals data, including accessing data related to the nutritional content of the purchased products. The retail server provides a discount to at least one of the purchased products if at least a combination of two or more items within the purchased products qualify as a medically tailored meal based on the nutritional content data surpassing a first threshold. Data relating to the purchased product is transmitted to the promotions system server upon completion.

### PRIORITY

This application claims the benefit of and priority to U.S. Provisional Application, entitled “Systems And Methods For Coupon-Based Incentive Promotions And Tracking,” filed on Sep. 30, 2019 and having application Ser. No. 62/908,500, the entirety of said application being incorporated herein by reference.

### FIELD

Embodiments of the present disclosure generally relate to the field of promotions. More specifically, embodiments of the disclosure relate to generating incentive-based promotions for specialized products with subsequent generated tracking metric data.

### BACKGROUND

Coupon-based promotions have traditionally been used to provide discounts to users for single products. These coupons have come in the form of a printed coupon that can be redeemed by a user in a retail store for a discount. More recently, digital coupons have allowed users to redeem coupons without the need for a printed coupon.

However, coupons have typically only applied to a single product or general class of products. Additionally, data related to the coupons and their redemption have typically not been thoroughly tracked by the issuer or other parties for health reasons. Third parties have increasingly desired to better track user behavior for various reasons. Insurance companies for example, often provide benefits and/or discounts to their customers for behavior that can lead to lower premiums and thus, increased profits.

What is needed, therefore, is a coupon system that can better utilize coupons to better track user behavior through multiple products and generate data that can be shared with third parties when needed.

### SUMMARY

A promotions system and methods are provided for generating incentive-based promotions for specialized products with subsequent generated tracking metric data. The promotions system is configured to receive promotion data, generate coupon data based on the promotion data, and transmit the coupon data to a retail server. The generated coupon data generally comprises approved products list data and medically tailored meals data. The retail server checks purchased products against the approved products list data and the medically tailored meals data, including accessing data related to the nutritional content of the purchased products. The retail server provides a discount to at least one of the purchased products if at least a combination of two or more items within the purchased products qualify as a medically tailored meal based on the nutritional content data surpassing a first threshold. Data relating to the purchased product is transmitted to the promotions system server upon completion.

In an exemplary embodiment, a promotions system server configured to increase customer purchases comprises: a processor; and a storage device commutatively coupled to the process, the storage device comprising: logic configured to be processed by the processor to: receive promotion data; generate a first set of coupon data based upon the promotion data; and transmit generated coupon data to a retail server; wherein the generated coupon data comprises approved products list data and medically tailored meals data.

In another exemplary embodiment, the retail server checks purchased products against the approved products list data. In another exemplary embodiment, the retail server checks purchased products against the medically tailored meals data. In another exemplary embodiment, checking the purchases against the medically tailored meals data comprises accessing data related to the nutritional content of the purchased products. In another exemplary embodiment, the retail server provides a discount to at least one of the purchased products if at least a combination of two or more items within the purchased products qualify as a medically tailored meal based on the nutritional content data surpassing a first threshold. In another exemplary embodiment, data relating to the purchased product is transmitted to the promotions system server upon completion.

In an exemplary embodiment, a method of providing promotions in conjunction with third parties comprises: receiving promotion data from a third party; generating a plurality of coupons based on the received promotion data wherein the plurality of coupons comprise: promotions on products related to a medically tailored meal; a promotional period with at least an end date; and at least one unique identifier associated with a single user of the coupon; mailing out the plurality of coupons in a package to the single user; receiving data associated with the redemption of the coupon and unique identifier; and mailing out a survey to the single user after the end date of the promotional period wherein the survey is customized based on the received data associated with the redemption of the coupon.

In another exemplary embodiment, the survey is provided to the single user as a digital communication. In another exemplary embodiment, a mid-promotion survey is provided to the single user prior to the end of the promotional period. In another exemplary embodiment, the method further comprises receiving response data from the single user in response to the mailed survey. In another exemplary embodiment, the received response data is shared with the third party.

### BRIEF DESCRIPTION OF THE DRAWINGS

The drawings refer to embodiments of the present disclosure in which:

FIG. 1 illustrates a system diagram of a digital incentive-based coupon system in accordance with an embodiment of the invention;

FIG. 2 illustrates a system diagram showing the connections between a server system and a retail system in accordance with one embodiment of the invention;

FIG. 3 illustrates a flow chart of processing incentive-based coupons in accordance with one embodiment of the invention; and

FIG. 4 illustrates a schematic representation of the components of a coupon-based management server in accordance with an embodiment of the invention.

While the present disclosure is subject to various modifications and alternative forms, specific embodiments thereof have been shown by way of example in the drawings and will herein be described in detail. The invention should be understood to not be limited to the particular forms disclosed, but on the contrary, the intention is to cover all modifications, equivalents, and alternatives falling within the spirit and scope of the present disclosure.

### DETAILED DESCRIPTION

The following description is not to be taken in a limiting sense, but is made merely for the purpose of describing the general principles of exemplary embodiments. The scope of the disclosure should be determined with reference to the claims. Reference throughout this specification to “one embodiment,” “an embodiment,” or similar language means that a particular feature, structure, or characteristic that is described in connection with the referenced embodiment is included in at least the referenced embodiment. Likewise, reference throughout this specification to “some embodiments” or similar language means that particular features, structures, or characteristics that are described in connection with the referenced embodiments are included in at least the referenced embodiments. Thus, appearances of the phrases “in one embodiment,” “in an embodiment,” “in some embodiments,” and similar language throughout this specification can, but do not necessarily, all refer to the same embodiment.

Further, the described features, structures, or characteristics of the present disclosure can be combined in any suitable manner in one or more embodiments. In the description, numerous specific details are provided for a thorough understanding of embodiments of the disclosure. One skilled in the relevant art will recognize, however, that the embodiments of the present disclosure can be practiced without one or more of the specific details, or with other methods, components, materials, and so forth. In other instances, well-known structures, materials, or operations are not shown or described in detail to avoid obscuring aspects of the present disclosure.

In the following description, certain terminology is used to describe features of the invention. For example, in certain situations, both terms “logic” and “engine” are representative of hardware, firmware and/or software that is configured to perform one or more functions. As hardware, logic (or engine) may include circuitry having data processing or storage functionality. Examples of such circuitry may include, but are not limited or restricted to a microprocessor, one or more processor cores, a programmable gate array, a microcontroller, a controller, an application specific integrated circuit, wireless receiver, transmitter and/or transceiver circuitry, semiconductor memory, or combinatorial logic.

Logic (or engine) may be software in the form of one or more software modules, such as executable code in the form of an executable application, an application programming interface (API), a subroutine, a function, a procedure, an applet, a servlet, a routine, source code, object code, a shared library/dynamic link library, or one or more instructions. These software modules may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; a semiconductor memory; non-persistent storage such as volatile memory (e.g., any type of random access memory “RAM”); persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the executable code is stored in persistent storage.

The term “processing” may include launching a mobile application wherein launching should be interpreted as placing the mobile application in an open state and performing simulations of actions typical of human interactions with the mobile application. For example, the mobile application, FACEBOOK®, may be processed such that the mobile application is opened and actions such as selecting to view a profile, scrolling through a newsfeed, and selecting and activating a link from the newsfeed are performed.

The term “mobile application” should be construed as a logic, software, or electronically executable instructions comprising a module, the mobile application being downloadable and installable on a network device. A mobile application may be a software application that is specifically designed to run on an operating system for a network device. Additionally, a mobile application may provide a graphical user interface (GUI) for the user of the network device.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

Coupon and other promotion systems have traditionally been used to incentivize behavior of users to facilitate an increased chance of purchasing a specific item related to the coupon. This coupon system was established by the makers of the products for sale and typically only generates communication between the product producer, the retailer, and in some instances an optional coupon clearing house. However, as technology has increased recently to better track users and sales, third parties have increasingly wanted to utilize coupon system to incentivize purchases and track data related to those purchases.

For example, insurance companies, including health insurance companies have provided programs to their customers in the past that grant benefits for participants. These programs have typically included requirements that their customers perform tasks or exhibit behaviors that raise the probability of increased profits by reducing potential insurance claims. As such, health insurance companies can often desire to track and incentivize what customers eat. Healthier eating habits are well known to correlate with better health, and thus a lower chance of medical problems requiring an insurance claim.

As a result, a coupon system can be utilized that is not run by or utilizes a product producer and can provide services related to behavior incentivizing, tracking, and subsequent data generation. In many embodiments, a third party may wish to incentivize users to eat medically tailored meals (“MTM”) which can consist of specific meals related to a diet that is associated with better health for the user. MTMs can comprise any of a variety of diets and can be generated in consultation with a medical professional or can be selected from a list of pre-generated MTMs. In many cases, an MTM meal requires a specific mix of nutritional information of multiple items. As such, traditional coupons would not be able to provide a discount on an MTM. However, methods and systems disclosed herein provide a method that utilizes computing devices to analyze nutritional info on a variety of products that are purchased to be able to verify if the individual products can be used in a MTM meal and/or diet and thus can be provided a discount. In some embodiments, MTM data is generated that corresponds to a specific type of diet. When a user is provided a coupon, the coupon can be for a specific product or a family or products that satisfies the requirements of an MTM. The MTM data can be general in nature (i.e., any user who is a member of the promotion and purchases products that qualify as an MTM will receive the discount). Additionally, the MTM data can be selective to specific users (i.e., a user who was given a specific MTM by their physician will only receive a discount if the products they purchase satisfy their specific MTM and not any other MTM).

This incentive-based coupon system can be utilized either within a digital system or via a printed coupon system with unique identifier numbers printed on each coupon. More specific detail related to these embodiments is given below.

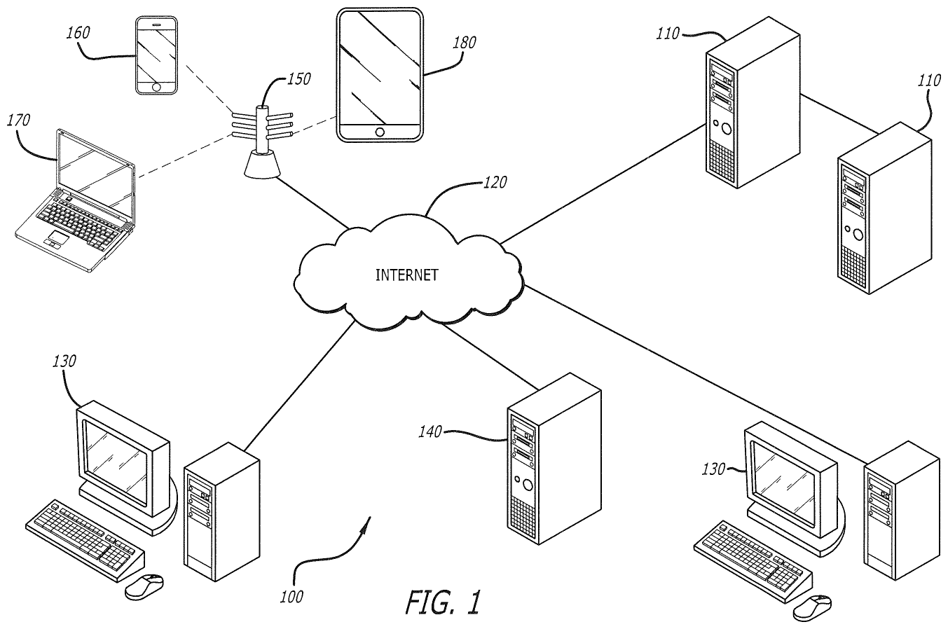

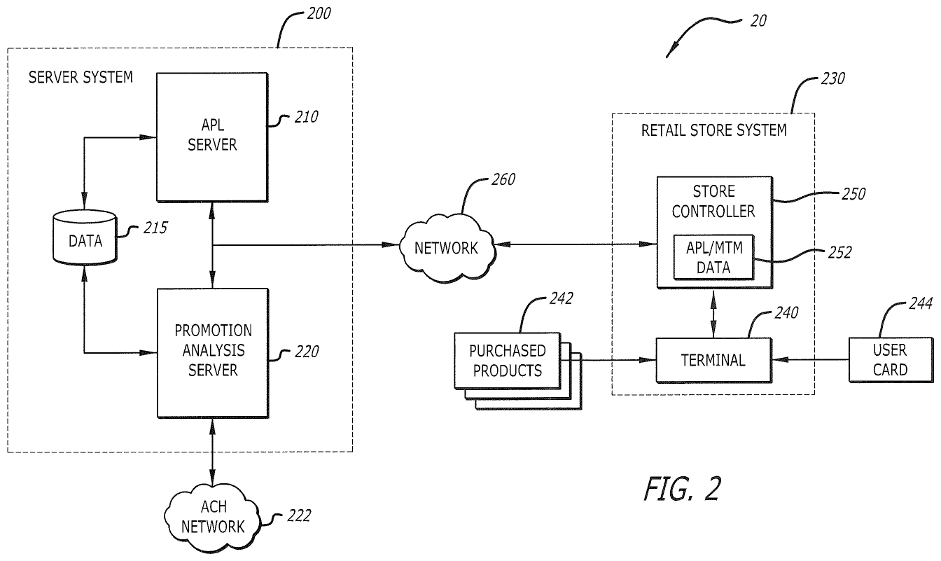

Referring to FIG. 1, a system diagram of a digital incentive-based coupon system in accordance with an embodiment of the invention is shown. The coupon management system 100 comprises a plurality of devices that are configured to transmit and receive data related to a variety of incentive-based coupons, products, and/or program metrics. In many embodiments, coupon management servers 110 are connected to a network 120 such as, for example, the Internet. Coupon management servers 110 are configured to transmit a variety of data across the network 120 to any number of receiving devices such as, but not limited to, personal computers 130, and mobile computing devices including laptop computers 170, cellular phones 160, and portable tablet computers 180. In many embodiments, coupon management system data may be transmitted to a retail server 140. The retail server 140 can be connected to a plurality of retail terminals that can process purchases of products that can be subject to a promotion, as further discussed below with respect to FIG. 2. In certain embodiments, the coupon management system data may be hosted on a cloud-based edge network system. In still additional embodiments, the coupon management servers 110 can be hosted as virtual servers within a cloud-based service.

In further embodiments, the sending and receiving of coupon management system data can occur over the network 120 through wired and/or wireless connections. In the embodiment depicted in FIG. 1, the mobile computing devices 160, 170, 180 are connected wirelessly to the network 120 via a wireless network access point 150. It should be understood by those skilled in the art that the types of wired and/or wireless connections between devices on the coupon management system 100 can comprise any combination of devices and connections as needed.

Referring now to FIG. 2, a system diagram showing the connection system 20 between a server system 200 and a retail system 230 over a network connection 260 in accordance with one embodiment of the invention is shown. The server system 200 is shown consisting of an approved product list (or “APL”) server 210 and a promotion analysis server 220, each of which may take the form of one or more computer systems. In certain embodiments, the APL server 210 and promotion analysis server 220 may be incorporated within the same server device and/or be implemented as software. Similarly, the retail store system 230 traditionally comprises at least one store terminal 240 and a store controller 250. In many embodiments, the retail store system 230 is similar to the retail store server 140 as depicted in FIG. 1. The terminal 240 may be a point of sale or electronic cash register (POS/ECR) computer system that processes products or other items 242 provided for purchase by a consumer. The store controller 250 communicates with each of the terminals 240 within a retail store environment and may also be responsible for managing external communication Such as over network 260.

In the communication system 20, the APL server 210 is responsible for developing and/or transmitting a list of approved products or MTM data for which promotions are managed by the server system 200. In other embodiments, the promotion data related to the APL and/or MTM data is transmitted to the store controller 250 prior to the start of the promotional period, where it can be utilized until the expiration date of the promotion. The approved product list (APL) and/or MTM data is transmitted to the retail store system 230 over the network 260 and can be saved as APL/MTM data 252. In many embodiments, this APL and/or MTM data can be created with help of a database 215. During a transaction in a retail store, a user presents their selected products 242 for purchase at the terminal. In some embodiments, physical coupons may be presented to the cashier to indicate that certain items should be checked against being part of the promotion. In a variety of embodiments, the user can present a type of user card 244 during the purchase which can indicate their participation in the promotion. By way of example and not limitation, a customer may enroll in the promotion with a store-based user loyalty card which is then checked against the store data system to verify their participation in the promotion. In other embodiments, the promotion may be utilized by any user and data gathered from the user card and/or coupons is logged and transmitted back to the server system 200 for metric tracking and future coupon promotional research.

In certain embodiments, the identifier on the user card 244 is utilized by the retail store system 230 (in either the terminal 240 or the store controller 250) to determine which products in the APL data 252 are relevant for the consumer presenting the card 244. Likewise, the retail store system 230 (in either the terminal 240 or the store controller 250) can also determine if at least one combination of items purchased qualify as a MTM based on the MTM data 252. In a number of embodiments, the products 242 presented for purchase can then be compared against a portion of the APL and/or MTM data 252 to create a filtered list of products being purchased that are potentially eligible for discounts. This list could then be sent by the retail store system 230 along with a user identifier (such as a card ID found on the user card 244) to the promotion analysis server 220 for verification and/or metric data storage.

In certain retail store environments, the ability to verify and process APL and/or MTM data may not be possible at the store controller 250 or terminal 240 level. In these cases, the promotion analysis server 220 can receive a purchased product data from the retail store system 230 and then examine the data base 215 to determine the whether the products and/or user are eligible for a current promotion. Data related to this processing can also then be stored as metric data. The calculated discounts can then be transmitted over the network 260 to the retail store system 230, where they are then applied to the purchase price of the products 242. In one embodiment, either or both of the credit and debit transactions can take place over the ACH network 222 accessed by the server system 200. Note that the actions of the APL server 210 and the promotion analysis server 220 need not be performed on physically distinct computer systems, nor need the terminal 240 and store controller 250 be physically distinct systems.

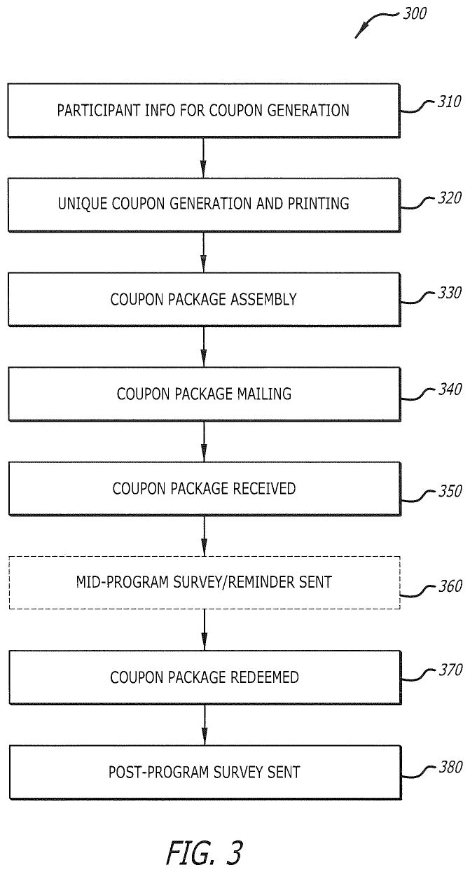

Referring now to FIG. 3, a flow chart of processing incentive-based coupons in accordance with one embodiment of the invention. In certain embodiments, physical coupons may be generated for users who are not otherwise able to participate in a digital promotion. In these instances, physical coupons can be printed and sent directly to the user. In a number of embodiments, the process 300 begins with participant information for the coupons to be printed are generated (block 310). This can typically be generated from user data within a database. In some embodiments, partnership with a third party product seller, employers, government agency, and/or insurance provider may utilize participant data provided by the third party. Once generated, unique coupons can be generated and subsequently printed (block 320). In a variety of embodiments, one-off printing can be utilized to generate unique coupons for each participant in the promotion. In this way, specific data markers can be embedded within the coupon for future verification and/or tracking purposes.

In many embodiments, this data encoding can be accomplished through GS1 barcodes printed on the coupons, with optional fields being utilized to store participant data. In some embodiments, the data pertaining to the participants may want to be shielded for reasons including, but not limited to, the Health Insurance Portability and Accountability Act (“HIPPA”). In these instances, the third party may provide blind user data (e.g., a serial number relating to a single user) to the promotion system and print the blind user data on the coupons to avoid matching specific blind data with a specific user. This blind data can be enhanced by outsourcing coupon printing and coupon packaging to separate third parties and/or providing the printed coupons to the third party to package and mail out themselves.

The printed coupons are then assembled (block 330) and mailed (block 340) to participants. In other embodiments, coupons can be packaged and sent to the third parties for distribution or to other marketing and/or retail businesses for direct consumer distribution. The coupon package is received 350, by the participants in the promotion. As stated above, the reception can be accomplished either through direct mailing or through distribution by other parties to the eventual retail customer.

In many embodiments, the coupon package can be tracked directly to the user receiving the coupons. In other embodiments, the coupon package may provide a means for users to log in and participate in the promotion such that their contact info is transferred or sent to a promotion administrator. In certain cases, the promotion may run over a particular duration of time. A retailer, product producer, and/or promotion administrator may seek to gain data relating to the promotion from the user before the promotion has ended. In these cases, an optional mid-program survey and/or reminder is sent to the participants of the promotion (block 360). The contact may include a reminder that the promotion period will be ending on a certain date. In other instances, the contact may request data on coupons were or were not used, and/or any feelings of the product and/or the user's wellbeing after purchase. Those skilled in the art will understand that surveys like this can include any of a variety of questions used to gather data relating to the promotion. Additionally, the contact can include any of a number of methods to elicit a response. These may include, but are not limited to, email messages, phone calls, in-app messages, push messages, and/or mailings.

During the promotional period, a particular number of users will redeem at least part of the coupon package (block 370). As discussed above, redemption can be made at the retail store level. In certain embodiments, redemption can be accomplished by entering a specialized code printed on the coupon within an online field of a website. In other embodiments, the coupon may be scanned by a camera on a smart phone to claim within a mobile phone application and/or website. In many embodiments, the coupons need to be redeemed physically. In virtual spaces this can be verified by utilizing one-off codes on each coupon so no coupon could be redeemed twice, or by use of specialized printing that is recognizable to scanning cameras that is not able to be replicated on home printing devices.

Finally, in response to the promotion expiring, a further post-program survey can be sent to any participants (block 380). The survey can be sent to participants who claimed the coupons and also those who did not. As discussed above, survey questions can be in any form that elicits valuable responses from the participants and can constitute any than that can be transmitted and utilized by a participant to properly replay and transmit data relating to their promotional experience back to the third party and/or promotional system administrator.

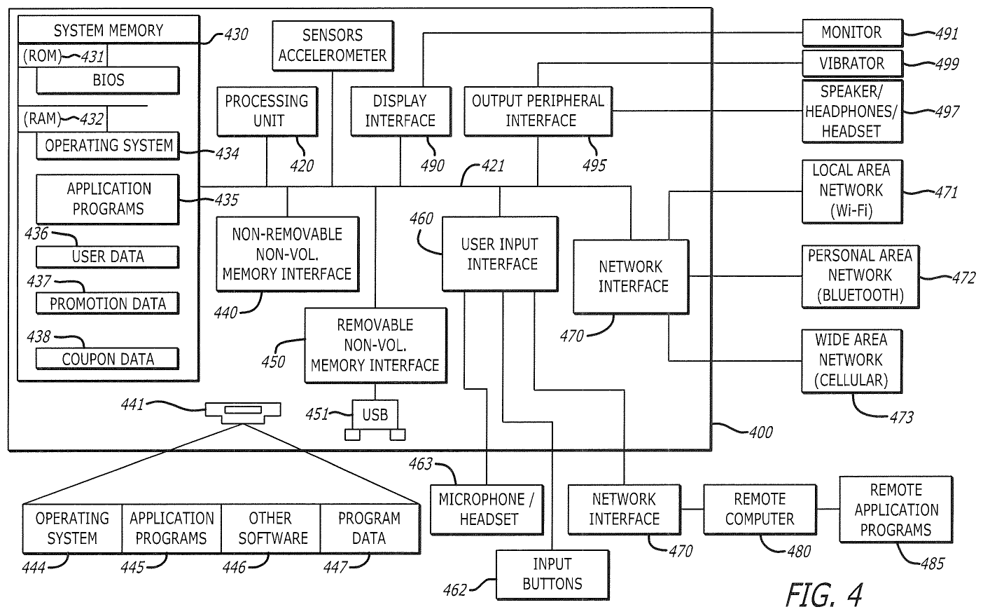

Referring now to FIG. 4, a schematic representation of the components of a coupon-based management system 400 in accordance with an embodiment of the invention is shown. The components of the coupon management system 400 can include, but are not limited to, a processing unit 420 having one or more processing cores, a system memory 430, and a system bus 421 that couples various system components including the system memory 430 to the processing unit 420. The system bus 421 can be any of several types of bus structures selected from a memory bus or memory controller, a peripheral bus, and a local bus using any of a variety of bus architectures. It will be understood by those skilled in the art that the coupon management system 400 can comprise any of a number of computing devices. By way of example and not limitation, the coupon management system 400 can be a coupon management server 110, a retail system 230, retail terminal 240, personal computer 130, cellular phones 160, laptop computer 170, or tablet computer 180.

The coupon management system 400 can include computing machine-readable media. The computing machine-readable media can be any available media that can be accessed by the coupon management system 400 and includes both volatile and non-volatile media, and removable and non-removable media. By way of example, and not limitation, computing machine-readable media use includes storage of information, such as computer-readable instructions, data structures, other executable software or other data. The computing machine-readable media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other tangible medium that can be used to store the desired information and that can be accessed by the computing device 400. Transitory media such as wireless channels are not included in the computing machine-readable media. Communication media typically embody computer-readable instructions, data structures, other executable software, or other transport mechanisms and include any information delivery media. As an example, some computing systems on a network might not have optical or magnetic storage.

The system memory 430 can include computing machine-readable media in the form of volatile and/or non-volatile memory such as read-only memory (ROM) 431 and random access memory (RAM) 432. A basic input/output system 433 (BIOS) containing basic routines configured for transferring information between elements within the coupon management system 400, such as during start-up, can be stored in the ROM 431. The RAM 432 can contain data and/or software immediately accessible to and/or presently being operated on by the processing unit 420. By way of example, and not limitation, FIG. 4 illustrates that the RAM 432 can include a portion of the operating system 434, the application programs 435, other executable software 436, and the program data 437.

The coupon management system 400 can also include other removable/non-removable volatile/nonvolatile computing machine-readable media. By way of example only, FIG. 4 illustrates a solid-state memory 441. Other removable/non-removable, volatile/nonvolatile computing machine-readable media that can be used in the example operating environment include, but are not limited to, USB drives and devices, flash memory cards, solid-state RAM, solid-state ROM, and the like. The solid-state memory 441 can be connected to the system bus 421 through a non-removable memory interface such as interface 440, and the USB drive 451 can be connected to the system bus 421 by a removable memory interface, such as interface 450.

The drives and their associated computing machine-readable media discussed above and illustrated in FIG. 4 provide storage of computer-readable instructions, data structures, other executable software and other data for the coupon management system 400. In FIG. 4, for example, the solid-state memory 441 is illustrated for storing operating system 444, application programs 445, other executable software 446, and program data 447. Note that these components can either be the same as or different from the operating system 434, the application programs 435, user data 436, promotion data 437 and coupon data 438. The operating system 444, the application programs 445, the other executable software 446, and the program data 447 are given different numbers here to illustrate that, at a minimum, they can be different copies.

A user (e.g., a customer, a retailer, a system administrator, etc.) can enter commands and information into the coupon management system 400 through input devices such as a keyboard, a touchscreen, software or hardware input buttons 462, a microphone 463, or a pointing device or scrolling input component such as a mouse, trackball, or touch pad. This input can be done directly on a coupon management system 400 or can be entered remotely on a client computing system 300 and transmitted directly as input to the coupon management system 400.

The coupon management system 400 can operate in a networked environment using logical connections to one or more remote computers/client devices, such as a remote computing system 480. The remote computing system 480 can be a cloud-based server, a personal computer, a hand-held device, a router, a peer device or other common network node, and can include many or all of the elements described above relative to the coupon management system 400. The logical connections depicted in FIG. 4 can include a personal area network (“PAN”) 472 (e.g., Bluetooth®), a local area network (“LAN”) 471 (e.g., Wi-Fi), and a wide area network (“WAN”) 473 (e.g., cellular network), but the logical connections can also include other networks. Such networking environments can be found in offices, enterprise-wide computer networks, intranets and the Internet. A browser application can be resident on the computing device and stored in the memory.

When used in a LAN networking environment, the coupon management system 400 can be connected to the LAN 471 through a network interface or adapter 470, which can be, for example, a Wi-Fi adapter. When used in a WAN networking environment (e.g., Internet), the coupon management system 400 typically includes some means for establishing communications over the WAN 473 such as the network interface 470. With respect to mobile telecommunication technologies, for example, a radio interface, which can be internal or external, can be connected to the system bus 421 via the network interface 470, or some other appropriate mechanism. In a networked environment, other software depicted relative to the coupon management system 400, or portions thereof, can be stored in a remote memory storage device. By way of example, and not limitation, FIG. 4 illustrates remote application programs 485 as residing on remote computing device 480. It will be appreciated that the network connections shown are examples and other means of establishing a communications link between the computing devices can be used.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. To the extent there are variations of the invention, which are within the spirit of the disclosure or equivalent to the inventions found in the claims, it is the intent that this patent will cover those variations as well. Therefore, the present disclosure is to be understood as not limited by the specific embodiments described herein, but only by scope of the appended

What is claimed is:

1. A promotions system server configured to increase customer purchases, the promotions system comprising:
a processor; and
a storage device commutatively coupled to the process, the storage device comprising:
logic configured to be processed by the processor to:
receive promotion data;
generate a first set of coupon data based upon the promotion data; and
transmit generated coupon data to a retail server;
wherein the generated coupon data comprises approved products list data and medically tailored meals data.

2. The promotions system of claim 1, wherein the retail server checks purchased products against the approved products list data.

3. The promotions system of claim 1, wherein the retail server checks purchased products against the medically tailored meals data.

4. The promotions system of claim 3, wherein checking the purchases against the medically tailored meals data comprises accessing data related to the nutritional content of the purchased products.

5. The promotions system of claim 4, wherein the retail server provides a discount to at least one of the purchased products if at least a combination of two or more items within the purchased products qualify as a medically tailored meal based on the nutritional content data surpassing a first threshold.

6. The promotions system of claim 1, wherein data relating to the purchased product is transmitted to the promotions system server upon completion.

7. A method of providing promotions in conjunction with third parties, the method comprising:
receiving promotion data from a third party;
generating a plurality of coupons based on the received promotion data wherein the plurality of coupons comprise:
promotions on products related to a medically tailored meal;
a promotional period with at least an end date; and
at least one unique identifier associated with a single user of the coupon;
mailing out the plurality of coupons in a package to the single user;
receiving data associated with the redemption of the coupon and unique identifier; and
mailing out a survey to the single user after the end date of the promotional period wherein the survey is customized based on the received data associated with the redemption of the coupon.

8. The method of claim 7, wherein the survey is provided to the single user as a digital communication.

9. The method of claim 7, wherein a mid-promotion survey is provided to the single user prior to the end of the promotional period.

10. The method of claim 7, further comprising receiving response data from the single user in response to the mailed survey.

11. The method of claim 10, wherein the received response data is shared with the third party.
